(19) United States
(12) Patent Application Publication
(10) Pub. No.: US 2010/0306905 A1
(43) Pub. Date: Dec. 9, 2010

(54) Detachable helmet visor

(76) Inventor: Peter J. Cornell, Beverly Hills, CA (US)

Correspondence Address: MARTIN & FERRARO, LLP, 1557 LAKE O'PINES STREET, NE, HARTVILLE, OH, 44632, US

(21) Appl. No.: 12/658,940

(22) Filed: Feb. 16, 2010

Related U.S. Application Data
(60) Provisional application No. 61/217,711, filed on Jun. 4, 2009.

(51) Int. Cl. A42B 1/24; A61F 9/00; A42B 1/08

(52) U.S. Cl. 2/422; 2/425; 2/10; 2/424

(57) ABSTRACT

A visor for use with a helmet. The visor includes a front panel and an elastic strap which form an internal perimeter configured to engage with the exterior of the helmet. The front panel includes an interior grip surface formed of a non-cloth material such as rubber.

### RELATED APPLICATIONS

This application claims the benefit of U.S. Provisional Application No. 61/217,711, filed Jun. 4, 2009, the disclosure of which is incorporated by reference herein.

### BACKGROUND OF THE INVENTION

1. Field of the Invention

The present invention relates to improvements in devices designed to protect from sunlight and snow the eyes, face and skin of a user wearing a helmet during outdoor activities.

2. Description of the Related Art

Helmets are used in various outdoor sporting activities including skiing, cycling, rafting and kayaking. A problem arises on sunny days when direct sunlight interferes with a user's ability to see properly. Protection from the overhead sunlight to shade the user's eyes is desirable to facilitate visibility during bright conditions. It is also desirable to protect the face from sunlight to prevent sunburn, skin cancer and aging changes in the face. It is furthermore a benefit to shield the eyes from falling snow to improve visibility in snow conditions.

Some helmets have molded visor components to provide some minimal shielding from the sun. Usually, such molded visors are integral to the helmet and are not detachable. If such visors are detachable from the helmet, they are configured to fit the specification of a particular helmet and are not useable with a variety of different helmets. Moreover, the assembly and disassembly of such visors from the helmet is not simple and the removed parts are not easily stored.

It is also common for skiers to wear goggles with a helmet when skiing. Goggles with tinted lenses can in part function to counteract the sunlight. However, the bright sunlight overhead can still interfere with a skier's vision. When a skier moves to shaded areas, the tinted lenses make it more difficult to properly see the surrounding terrain.

A further problem is typically encountered in ski resorts when it is snowing. For example, when a skier is on a ski lift or elsewhere on the slopes, the falling snow will accumulate on the lenses of the goggles and cause the user to frequently clear the lenses to be able to see through the goggles.

Accordingly, there is a need for eye protection suitable for use in both sunny and shaded areas; that is easily usable with various sizes and shapes of helmets; that is simply and easily attachable and detachable to such helmets; that is easily stored; and/or can assist in sheltering the goggles of a user from falling snow.

### SUMMARY OF THE INVENTION

The present invention in one preferred embodiment is drawn to a detachable visor to be worn over a sports helmet that is designed to attach snugly to a helmet with the back section being positioned in the goggle depression in the back of the helmet and the front section designed with elastic pressure and physical design to fit on the front of the helmet, and a brim or bill to protect the wearer's eyes from sunlight while taking part in outdoor activities.

In another preferred embodiment, the present invention applies to the use of a visor to protect the eyes from sunlight which is applied to helmets, such as helmets worn during outdoor activities like skiing. The visor is detachable and can be placed on the helmet and removed easily as weather conditions change.

In one preferred embodiment, the present invention uses a visor with an elastic band to snug the visor to the helmet. The back strap portion of the visor will fit into the depression in current helmets that is designed for goggles, and is thin enough to fit in addition to goggles if desired. A retention strap in current helmet designs can be used to secure the back strap portion of the visor.

The front portion of the visor is preferably made to snugly fit with elastic pressure, design of the band, and possible with a tacky interior material above the goggle opening of the helmet such as to allow the brim of the visor to extend over the front lower edge of the helmet and shield the eyes of the wearer from sunlight.

The brim can come in a variety of lengths, styles, and the overall visor can be in a variety of materials including cloth or plastic or any other material suitable for its intended purpose. The visor and brim can have a variety of colors, patterns, logos, and the like, much as golf visors do. The visor may be made of any material suitable for the intended purpose, including traditional cotton or a waterproof material.

The front of the visor may have a different circumference, with the lower circumference greater than the upper such as to snugly fit certain helmet designs.

The front of the visor may be made of a different radius of curvature on the top and bottom of the visor such that it will fit securely on the front of the helmet.

The inner material of the front of the visor may be made of a material that is slip resistant such that the visor will stay securely on the helmet during activity.

The material of the visor may be made out of a waterproof material for wet conditions.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE FIGURES

FIG. 1 is a perspective view of a visor in accordance with a preferred embodiment of the present invention shown engaged with a helmet.

FIG. 2 is a side elevation view of the visor and helmet of FIG. 1.

FIG. 3 is a bottom plan view of the visor of FIG. 1.

FIG. 4A is a partial cross sectional view of the visor and helmet taken along line 4-4 of FIG. 2.

FIG. 4B is a partial cross sectional view of an alternative embodiment of the visor having lip for engagement with the helmet.

FIG. 5 is an expanded view of the gripping surface taken along line 5 of FIG. 3.

### DETAILED DESCRIPTION OF THE DRAWINGS

Reference will now be made in detail to the present preferred embodiments of the invention, examples of which are illustrated in the accompanying drawings.

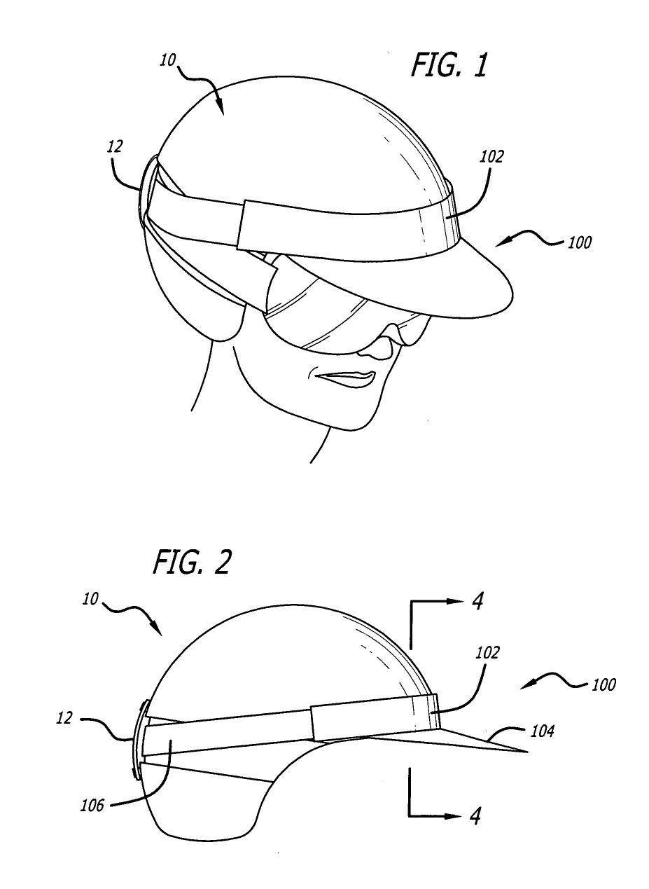

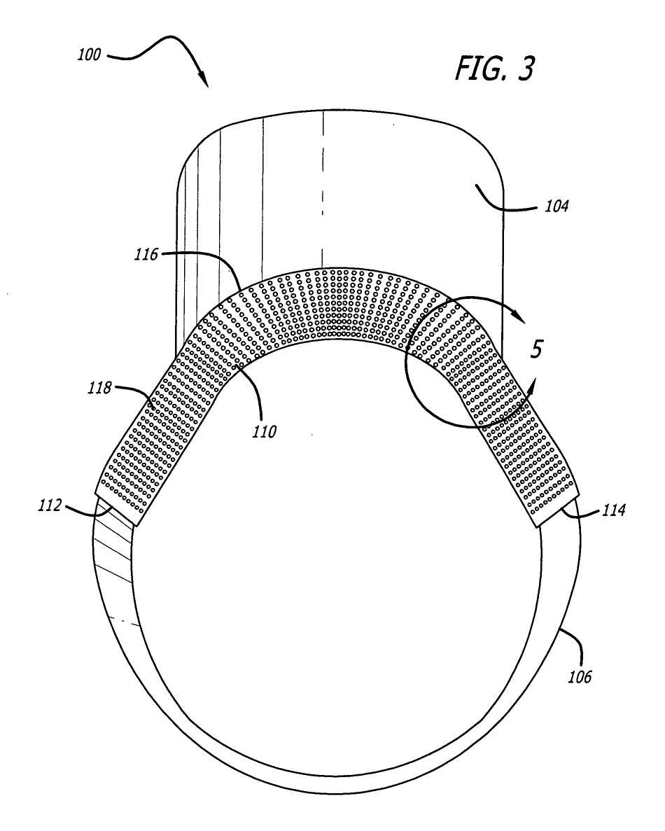

FIGS. 1 to 5 show a preferred embodiment of a detachable helmet visor 100 engageable with a helmet 10. In a preferred embodiment, visor 100 includes a front panel 102, a bill 104, and an elastic strap 106. The visor is preferably configured with an enlarged internal diameter and non-slip interior gripping surface so that it snugly engages with helmet 10 to provide a user additional protection of his or her eyesight. The preferred elements of and their interrelationship are described below.

Referring to FIGS. 2 to 4A, front panel 102 of visor 100 includes an exterior surface 108, an opposite interior grip surface 110, first and second ends 112, 114, respectively, and a base 116 along the length of front panel 102. In a preferred form, front panel 102 is generally rectangular.

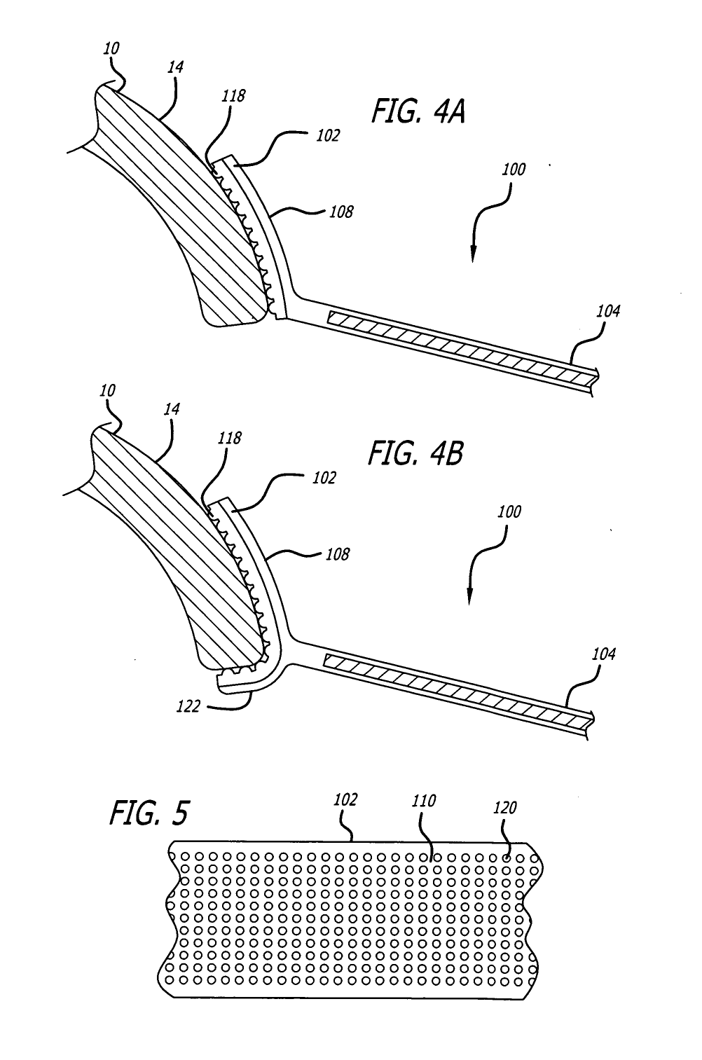

FIG. 4B shows an alternative embodiment of the visor that preferably includes a lip 122 arranged along a substantial portion of the length of base 116 of front panel 102. Lip 122 is preferably configured to engage the edge of helmet 10. Lip 122 may be configured for stiff or resilient engagement with the helmet. Instead of, or in addition to lip 122, a thin ridge of material may be included proximate base 116 along interior grip surface 110. Such a ridge may be approximately 0.25 inches thick and made from the same or different material as grip surface 110. Preferably, the ridge is configured to allow the visor to ride over the edge of helmet 10, thereby providing the visor with an improved ability to hold onto the helmet at higher speeds.

As shown in FIG. 5, grip surface 110 preferably includes a surface texture or roughenings 118. Roughenings 118 preferably include a plurality of projections 120. FIG. 5 shows projections 120 generally configured in a grid pattern. It will be appreciated that projections 120 may vary in number, shape and configuration without departing from the scope of the present invention. Surface roughenings may be omitted if desired. For example only, grip surface 110 may be completely smooth and have a non-slip characteristic. It is preferred that grip surface 110 have a characteristic sufficient to maintain visor 100 from slipping against exterior surface 14 of helmet 10 when the visor is engaged thereto.

Grip surface 110 preferably is made of a non-cloth material such as a plastic, PVC, silicon or rubber, which assists in preventing slippage of front panel 102 relative to exterior surface 14 of helmet 10. It will be appreciated that a variety of materials may be used to enhance the ability of grip surface 110 to grip exterior surface 14 of helmet 10. While grip surface 110 is preferably made of a non-cloth material, the rest of the visor may be made of or include cloth materials such as polyester.

Preferred dimensions are set forth below, although it will be appreciated that the dimensions may be varied as suitable for the intended application. Front panel 102 and strap 106 preferably form a generally circular perimeter having a minimum diameter of at least 22 cm in an unstretched state. This preferably results in a diameter that is typically larger than the diameter of an average human head. Front panel 102 preferably has a length of approximately 36 cm along base 116 of the front panel. Strap 106 preferably has a length of approximately 37 cm. Bill 104 preferably has a length of approximately 8 cm measured from where it intersects front panel 102 to the front-most portion of the bill. Grip surface 110 preferably has a thickness of approximately 1.5 to 2.0 mm, more preferably 1.8 mm. It will be appreciated that these dimensions are representative only and may be varied as appropriate depending on, for example, the intended application and material. For example, the length of the bill may be varied considerably depending upon the particular activity or helmet envisioned for the ultimate use. It is preferred that the interior diameter of the visor be greater than the average head size of an adult human. Although the term “diameter” is used to describe the interior spacing of the strap and front panel, it will be appreciated that the interior spacing need not form a perfect circle. For example, the interior perimeter of the strap and front panel in an unexpanded state may resemble an oval or ellipse if desired.

Having described the preferred components of visor 100, a preferred method of use will now be described with reference to FIGS. 1 and 2. To engage visor 100 to helmet 10, a user preferably expands elastic strap 106 of the visor around the circumference of the helmet; positions front panel 102 across the front portion of helmet 10; and maintains the front panel in a fixed position relative to the front portion of the helmet. The maintenance of the front panel with the helmet may be by way of gripping surface 118 and/or other means such as interlocking projections, snaps, and one or more protruding lips (see, e.g., FIG. 4B). Strap 106 may be engaged with hook 12 of helmet 10 to provide further security of the attachment of visor 100 with helmet 10. Preferably, the visor is engaged with the helmet without adjusting the strap with a secondary device, such as a clip or buckle. Preferably, the visor is engaged with a strap instead of being clipped or snapped into place onto a helmet. Preferably the strap has no folds when it is engaged to the helmet.

It will be appreciated that the steps described above may be performed in a different order, varied, or some steps omitted entirely without departing from the scope of the present invention.

The foregoing description is by way of example only, and may be varied considerably without departing from the scope of the present invention. For example only, the visor may be combined with a helmet and sold as a kit. The helmet may be specially designed for use with the visor, for example, with a groove or indentation for the visor strap, which may be oriented at an angle in relation to any groove for goggles. More than one hook or fastener may be used to add further securement of the visor to the helmet. The bill may be made of a translucent or transparent (clear or tinted) material. The strap may be formed as a single strap (as shown in FIG. 1), or a plurality of smaller straps. The width of the strap may vary as needed for a particular application.

Supplemental attachment means may be included as part of the visor. For example, portions of the front panel and/or strap may include hook and loop fasteners such as Velcro, or snaps. The strap may include engagement members, such as clips or snaps, to assist in securely retaining the strap to the helmet. The strap may include grip material on its interior facing surface to assist in keeping the strap from moving substantially when engaged with the exterior of the helmet. Such grip material may be made from silicon, rubber and/or another material suitable for the intended purpose, and configured as a series of dots, squares, or a strip extending around a portion of the inside perimeter of the strap.

The visor may be specifically designed to accommodate a helmet that is sport-specific, such as cycling, motorbike, and/or skiing. The visor may be used in a variety of other activities where supplemental eye protection would be beneficial, such as in a military environment (e.g., with a military helmet and/or goggles).

The present invention in a preferred form provides the advantages of additional eye protection in adverse weather environments and sunny days.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A visor for use with a helmet, comprising:
a front panel having an exterior surface, an opposite interior grip surface, first and second ends, and a base, said interior grip surface comprising a non-slip material configured to provide friction against a surface of the helmet to prevent sliding of the visor relative to the helmet;
a bill extending from a portion of said base; and
an elastic strap fixed to each end of said front panel.

2. The visor of claim 1, wherein said interior surface of said front panel includes a plurality of surface roughenings.

3. The visor of claim 1, wherein said interior surface of said front panel is smooth.

4. The visor of claim 1, further comprising a lip extending from said base, said lip being adapted to engage a rim of the helmet when said visor is applied to the helmet.

5. The visor of claim 1, wherein said elastic strap has an interior surface with a length that includes hook and loop fasteners along at least a portion thereof.

6. The visor of claim 1, in combination with a helmet.

7. The visor of claim 1, in combination with a ski helmet.

8. The visor of claim 1, in combination with a bike helmet.

9. A visor for use with a helmet, comprising:
a front panel having an exterior surface, an opposite interior surface, first and second ends, and a base;
a bill extending from a portion of said base; and
a single elastic strap fixed to said first and second ends of said front panel, said front panel and said elastic strap forming a generally circular perimeter with a minimum diameter of at least 22 cm in an unstretched state, said strap being stretchable to be placed around at least a portion of a circumference of the helmet.

10. The visor of claim 9, wherein said interior surface of said front panel is made of a rubber material.

11. The visor of claim 9, wherein said interior surface of said front panel includes a plurality of surface roughenings.

12. The visor of claim 9, wherein said interior surface of said front panel is smooth.

13. The visor of claim 9, further comprising a lip extending from said base, said lip being adapted to engage a rim of the helmet when said visor is applied to the helmet.

14. The visor of claim 9, wherein said elastic strap has an interior surface with a length that includes hook and loop fasteners along at least a portion thereof.

15. The visor of claim 9, in combination with a helmet.

16. The visor of claim 9, in combination with a ski helmet.

17. The visor of claim 9, in combination with a bike helmet.

18. A method for engaging a visor to a helmet of a user, comprising:
providing the visor having a front panel, a bill and an elastic strap;
providing the helmet having an external circumference, with a front portion and a rear portion, the rear portion including a hook;
expanding the elastic strap of the visor around the circumference of the helmet;
positioning the front panel across the front portion of the helmet; and
maintaining the front panel in a fixed position relative to the front portion of the helmet.

19. The method of claim 18, wherein the maintaining of the front panel in a fixed position includes maintaining the front panel without sliding the front panel relative to the front portion of the helmet when encountering wind resistance while the user is in motion.

20. The method of claim 18, further comprising securing the strap to the helmet with the hook.
